(19) United States
(12) Patent Application Publication
(10) Pub. No.: US 2025/0082461 A1
(43) Pub. Date: Mar. 13, 2025

(54) Medical implant for filling a cavity in a glandular tissue and method of manufacturing a medical implant for filling a cavity in a glandular tissue

(71) Applicant: pfm medical gmbh, Koeln (DE)

(72) Inventor: Monika Rieck, Koeln (DE)

(21) Appl. No.: 18/816,497

(22) Filed: Aug. 27, 2024

(30) Foreign Application Priority Data
Sep. 12, 2023 (IT) 102023000018666

(51) Int. Cl. A61F 2/12; A61L 27/06; A61L 27/16; A61L 27/30

(52) U.S. Cl. CPC A61F 2/12; A61L 27/06; A61L 27/16; A61L 27/306; A61F 2240/001; A61L 2430/04

(57) ABSTRACT

The invention relates to a medical implant (1) for filling a cavity in a glandular tissue, in particular in the glandular tissue of the human breast, wherein the medical implant (1) consists of a titanized polymer and is designed as a mesh to cover the cavity to be filled. The invention also relates to a method for manufacturing the medical implant (1).

### CROSS REFERENCE TO RELATED APPLICATION

The present application claims priority to Italian Patent Application No. 102023000018666 filed Sep. 12, 2023. the disclosure of which is incorporated herein by reference.

### BACKGROUND

The invention relates to a medical implant for filling a cavity in a glandular tissue, in particular in the glandular tissue of the human breast. The invention also relates to a method for manufacturing a medical implant for filling a cavity in a glandular tissue.

The implant according to the invention is used in particular to fill a cavity in the human breast after breast-conserving breast cancer surgery. In breast-conserving surgery, the surgeon completely removes the tumor tissue from the breast tissue (glandular tissue). The tumor is removed with the smallest possible margin of surrounding healthy tissue. The margins (cut edges) of the removed tissue and/or the tumor bed (area of the breast where the tumor was located) are examined for cancer cells in the laboratory after the operation. Newer methods also allow the edges of the removed tissue to be examined during the operation. If the histological examination reveals that the cancerous tissue has not been completely removed, it must be recut during the operation or a new operation must be performed to recut the tissue.

However, removing the tumor from the breast tissue creates a cavity that may be visible from the outside. This cavity is preferably filled so that it is not visible from the outside. However, small implants in particular often lead to encapsulation, inflammation, growths or even rejection.

An alternative to breast-conserving surgery for breast cancer is the complete removal of the breast (mastectomy). The removed breast can subsequently be reconstructed (breast reconstruction). To this end, WO2013/148719 A1, for example, discloses a mesh implant for holding a breast implant. The mesh implant is fixed to the pectoral muscle so that the implant cannot slip. To improve biocompatibility, the mesh implant is preferably coated, in particular titanized.

Based on this state of the art, the invention is based on the task of providing a medical implant for filling a cavity in a glandular tissue and at the same time preventing encapsulation, inflammation, proliferation or rejection reactions.

The problem is solved according to the invention by a medical implant for filling a cavity in a glandular tissue, in particular in the glandular tissue of the human breast, wherein the medical implant consists of a titanized polymer and is formed as a mesh to cover the cavity to be filled.

The mesh structure of the medical implant according to the invention ensures that the surrounding glandular tissue (breast tissue) can grow into the mesh structure. At the same time, the use of a titanized polymer prevents the formation of encapsulations, inflammations, growths or rejection reactions. The medical implant according to the invention thus generates growth of the surrounding tissue, in particular the surrounding tumor bed, into the mesh structure of the implant, whereby the cavity is filled by the patient's own tissue. Due to the biocompatibility of the titanized polymer, this newly grown tissue does not contain any encapsulations or growths, which are caused in particular by inflammatory processes or rejection reactions.

A titanized polymer within the meaning of the invention is a polymer fibre which is coated with titanium. Conveniently, the titanium coating is applied so thinly that the elasticity and flexibility of the polymer fiber is not restricted. Preferably, the medical implant is made of a polymer mesh which is subsequently titanized. This has the advantage that the subsequently applied titanium coating fixes the mesh structure so that the medical implant can be cut to size at a later time without the mesh structure dissolving.

According to an expedient variant of the invention, the medical implant consists of a titanized polypropylene.

In an advantageous variant of the invention, the medical implant has the shape of a plus sign (+). The intersection of the two legs of the plus sign can be positioned at the deepest point of the cavity to be filled, so that the legs extending therefrom at least partially, preferably completely, cover the side walls of the cavity to be filled. If parts of the legs protrude from the cavity to be filled, these can be cut off after the medical implant according to the invention has been inserted. In this form, the implant according to the invention can be adapted particularly easily to the size, in particular the depth, of the cavity to be filled.

According to a variant of the invention, the mesh has a mesh width of less than 2 mm, preferably a mesh width of 1 mm. This mesh size is particularly advantageous for the ingrowth of the surrounding tissue, in particular the glandular tissue or breast tissue.

According to an expedient variant of the invention, the medical implant has a weight of less than 50 g/m², in particular 16 g/m² or 35 g/m².

In a particularly useful variant of the invention, the thickness of the titanium coating is selected such that the elasticity and flexibility of the polymer mesh is not impaired. For example, the polymer fiber of the mesh has a diameter of 50 to 100 μm and the titanium coating has a thickness in the range of 15 to 75 nm.

The problem is further solved by a method for manufacturing a medical implant according to the invention comprising the steps:
providing a mesh made of a titanized polymer, the dimensions of the mesh being larger than the medical implant to be manufactured,
cutting out areas of the provided mesh to obtain the desired shape of the medical implant, the obtained shape of the desired implant remaining connected to the offcut of the provided mesh at predefined locations.

The medical implant according to the invention is thus a part of the provided mesh with larger dimensions than the actual medical implant until shortly before the actual implantation. Only immediately before implantation are the predefined connection points separated, whereby the medical implant is released from the surrounding mesh structure. This improves the manageability of the medical implant until it is actually implanted.

In a preferred variant of the method according to the invention, at least two medical implants of different sizes are cut out in the mesh provided. Thus, several medical implants of different sizes are provided in the mesh structure and the surgeon can detach the medical implant from the mesh during the operation, which corresponds to the size of the cavity to be filled. As already mentioned at the beginning, the tumor tissue is removed together with a margin of the surrounding tissue. It is not possible to predict in advance exactly how large the cavity to be filled will be, especially if it is necessary to recut during the operation because, for example, an examination of the fine tissue during the operation has revealed that not all of the tumor tissue has been removed. By cutting several medical implants of different sizes from the mesh, the surgeon can select the medical implant that is the ideal size for the cavity to be filled by separating the defined connection points. Furthermore, the surgeon can test the fit of the different medical implants in the cavity to be filled before deciding on a medical implant. Providing several medical implants of different sizes in one mesh is more economical than providing separate meshes with one medical implant each, as these would have to be individually packed, sterilized, stored, etc.

According to an appropriate variant of the invention, the method comprises the step of packaging and sterilizing the provided mesh and the at least one medical implant cut out therein.

According to an advantageous variant of the invention, the titanized mesh is produced by:
providing a polymer mesh, and
applying a titanium layer to the provided polymer mesh.

The subsequent application of the titanium layer fixes the mesh structure better, so that cutting the mesh has no negative effect on the mesh structure.

In a particularly useful variant of the invention, the thickness of the titanium coating is selected such that the elasticity and flexibility of the polymer mesh is not impaired. For example, the polymer fiber of the mesh has a diameter of 50 to 100 μm and the titanium coating has a thickness in the range of 15 to 75 nm.

### BRIEF DESCRIPTION OF THE DRAWINGS

The invention is explained in more detail below with reference to embodiments shown in the figures. They show:

FIG. 1 schematic view of a medical implant according to the invention,

FIG. 2 schematic view of a titanized polymer mesh with two medical implants according to the invention cut out therein, and

FIG. 3 a flow chart of a method according to the invention for manufacturing a medical implant according to the invention.

### DETAILED DESCRIPTION

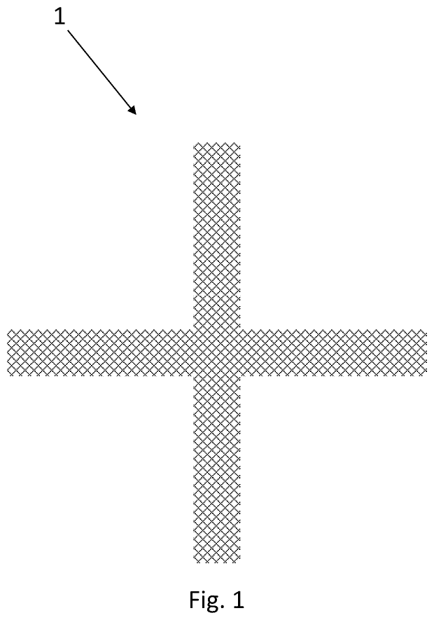

FIG. 1 shows a schematic view of a medical implant 1 according to the invention for filling a cavity in a glandular tissue, in particular in the glandular tissue of the human breast. The medical implant 1 of FIG. 1 consists of a titanized polymer and is designed as a mesh to cover the cavity to be filled.

Because the medical implant 1 according to the invention is designed as a mesh and covers the cavity to be filled in the implanted state, the surrounding glandular tissue (breast tissue) can grow into the mesh structure of the medical implant 1. At the same time, the use of a titanized polymer prevents the formation of encapsulations, inflammations, growths or rejection reactions. The medical implant 1 according to the invention promotes growth of the surrounding tissue, which is covered by the medical implant 1 in the implanted state, into the mesh structure of the medical implant 1. The generated growth of the surrounding tissue fills the cavity with the patient's own tissue. The biocompatibility of the titanized polymer prevents encapsulation or growths in the newly grown tissue, which are caused in particular by inflammatory processes or rejection reactions.

A titanized polymer within the meaning of the invention is a polymer fibre which is coated with titanium. Conveniently, the titanium coating is applied so thinly that the elasticity and flexibility of the polymer fiber is not restricted. Preferably, the medical implant 1 is made from a polymer mesh which is subsequently titanized. This has the advantage that the subsequently applied titanium coating fixes the mesh structure so that the medical implant 1 can be cut to size at a later time without the mesh structure dissolving.

Preferably, the medical implant 1 consists of a titanized polypropylene. The mesh structure of the medical implant 1 preferably has a mesh size of less than 2 mm, in particular a mesh size of approximately 1 mm. The medical implant 1 preferably has a weight of less than 50 g/m², in particular 16 g/m² or 35 g/m².

As shown in FIG. 1, the medical implant 1 has the shape of a plus sign, or an X-shape after a rotation of 45°. The intersection of the two legs of the plus sign is positioned at the deepest point of the cavity to be filled during implantation of the medical implant 1 and the legs extending from it cover the side walls of the cavity to be filled at least partially, preferably completely. In order to adapt the implant according to the invention particularly easily to the size, in particular the depth, of the cavity to be filled, the parts of the legs that protrude from the cavity to be filled can be cut off.

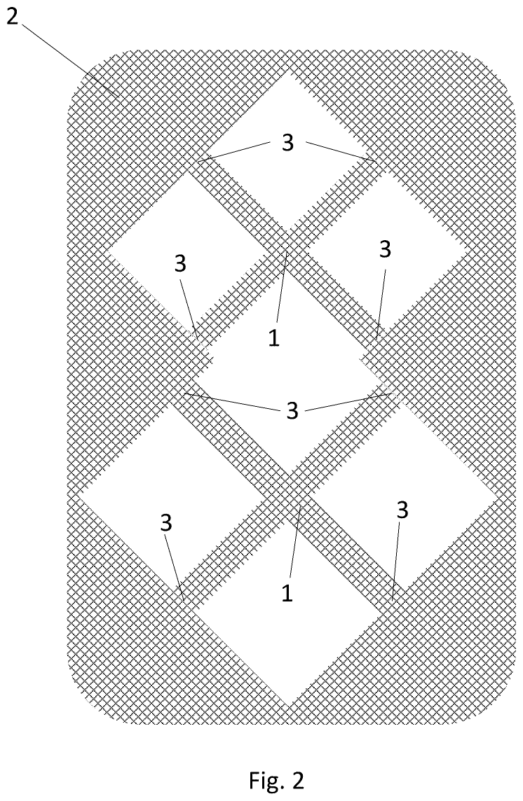

FIG. 2 shows a schematic view of a titanized polymer mesh 2 with two medical implants 1 according to the invention cut out therein. The two medical implants 1 are connected to the mesh 2 at predefined points 3 and can be detached (cut) from the mesh 2 before implantation.

The two medical implants 1 cut out in the mesh 2 have different sizes, so that the surgeon can select the medical implant 1 in the appropriate size during the operation. The surgeon can also compare the fit of the two medical implants 1 with each other and determine the appropriate size of the medical implant 1 on this basis.

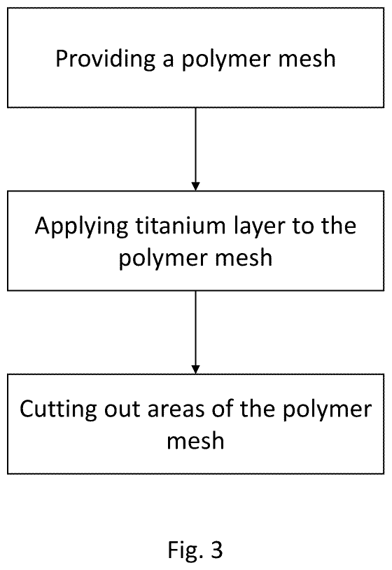

FIG. 3 shows a flow chart of a method according to the invention for manufacturing a medical implant 1 according to the invention. In a first step, a mesh 2 of polymer fibers is provided. For example, the polymer fibers have a diameter of 50 to 100 μm. The dimensions of the mesh 2 are larger than the medical implant 1 to be produced or the medical implants 1 to be produced therefrom.

In the next step, a titanium layer is applied to the prepared mesh. The titanium layer has a thickness in the range of 15 to 75 nm, for example. Conveniently, the titanium layer applied does not impair the elasticity and flexibility of the polymer mesh 2.

In the final step of the manufacturing process, areas of the prepared and titanized mesh 2 are cut out in order to obtain the desired shape of the medical implant 1. As shown in FIG. 2, several medical implants 1 of different sizes can also be cut out of the mesh 2.

The resulting shape of the desired implant 1 remains connected to the offcut of the provided mesh 2 at predefined points 3.

Finally, the prepared mesh 2, including the medical implant 1 cut out of it, can be packaged and sterilized. In this state, the packaged mesh 2 is transported and stored until it is inserted into the cavity to be filled by a surgeon in a breast-conserving operation. During the operation, the surgeon can detach the medical implant 1 from the surrounding mesh 2 by separating, in particular cutting, the predefined areas.

### LIST OF REFERENCE SYMBOLS

1—medical implant
2—mesh
3—predefined connection point

1. A medical implant (1) for filling a cavity in a glandular tissue, in particular in the glandular tissue of the human breast,
wherein the medical implant (1) is made of a titanized polymer and is formed as a mesh to cover the cavity to be filled.

2. A medical implant (1) according to claim 1, wherein the medical implant (1) consists of a titanized polypropylene.

3. A medical implant (1) according to claim 1 or claim 2, wherein the medical implant (1) has the shape of a plus sign.

4. A medical implant (1) according to any one of claims 1 to 3, wherein the mesh has a mesh size of less than 2 mm, preferably a mesh size of 1 mm.

5. A medical implant (1) according to any one of claims 1 to 4, wherein the medical implant (1) has a weight of less than 50 g/m², in particular of 16 g/m² or 35 g/m².

6. A method of manufacturing a medical implant (1) according to any one of claims 1 to 5, comprising the steps of:
providing a mesh (2) of a titanized polymer, wherein the dimensions of the mesh (2) being larger than the medical implant (1) to be manufactured,
cutting out areas of the provided mesh (2) to obtain the desired shape of the medical implant (1), wherein the obtained shape of the desired implant (1) remaining connected to the offcut of the provided mesh (2) at predefined locations (3).

7. A method according to claim 6, wherein at least two medical implants (1) of different sizes are cut out in the provided mesh (2).

8. A method according to claim 6 or claim 7, comprising the step of packaging and sterilizing the provided mesh (2) and the at least one medical implant (1) cut out therein.

9. A method according to any one of claims 6 to 8, wherein the titanized mesh (2) is produced by:
providing a polymer mesh, and
applying a titanium layer to the provided polymer mesh.

10. A method according to claim 9, wherein the thickness of the titanium layer is selected such that the elasticity and flexibility of the polymer network is not impaired.
